(19) United States
(12) Patent Application Publication
(10) Pub. No.: US 2023/0330059 A1
(43) Pub. Date: Oct. 19, 2023

(54) 3-PHENYL-2H-CHROMENE DERIVATIVE AND PHARMACEUTICAL COMPOSITION FOR PREVENTING OR TREATING ALZHEIMER'S, CONTAINING SAME

(71) Applicant: NEUROBIONET, INC., Gyeonggi-do (KR)

(72) Inventors: Seong Soo AN, Gyeonggi-do (KR); Sunyeou KIM, Seoul (KR); Raok JEON, Seoul (KR); Keun-A CHANG, Seoul (KR); Kyu Hwan SHIM, Gyeonggi-do (KR); Seongmin HONG, Gyeonggi-do (KR); Jaehyuk LEE, Incheon (KR); Dahye YOON, Gyeonggi-do (KR); Hyewon CHO, Seoul (KR); Shinwoo KANG, Incheon (KR); Hyunjun PARK, Gyeonggi-do (KR); Chandra Bhushan MISHRA, Seoul (KR)

(73) Assignee: NEUROBIONET, INC., Gyeonggi-do (KR)

(21) Appl. No.: 18/028,816

(22) PCT Filed: Sep. 1, 2021
(86) PCT No.: PCT/KR2021/011746

§ 371 Date: Mar. 28, 2023

(30) Foreign Application Priority Data
Sep. 29, 2020 (KR) 10-2020-0127557

(51) Int. Cl. A61K 31/352; A61P 25/28

(52) U.S. Cl. CPC A61K 31/352; A61P 25/28

(57) ABSTRACT

The present invention relates to a 3-phenyl-2H-chromene derivative and a pharmaceutical composition for preventing or treating Alzheimer’s, containing same as an active ingredient. The compound, a solvate thereof, a hydrate thereof, or a pharmaceutically acceptable salt thereof, provided according to one aspect of the present invention, inhibits, with high activity, the fibrosis and oligomerization of Aβ and thus has the effect of being usefully employable for preventing or treating Alzheimer’s.

### TECHNICAL FIELD

The present disclosure relates to a 3-phenyl-2H-chromene derivative and a pharmaceutical composition containing same as an active ingredient for prevention or treatment of Alzheimer’s disease.

### BACKGROUND ART

Neurodegenerative diseases collectively refer to neurological diseases that occur as the structures and functions of the central nervous system and the peripheral nervous system are progressively lost over time. The highest in incidence among them is Alzheimer’s disease. Although the exact cause of Alzheimer’s disease is not yet clearly known, it is presumed that amyloid plaques transform normal Alzheimer’s proteins to form plaque masses and lose their original functions. Alzheimer’s disease has histopathological traits including general atrophy of the brain, dilatation of the ventricles, multiple lesions of nerve fibers, and aging spots.

Among the several major pathological symptoms occurring in Alzheimer’s disease, the cytotoxicity of cholinergic neurons due to aggregation of amyloid beta (Aβ) is known to be the main cause of Alzheimer’s, and deposition/accumulation of Aβ is the cause of irreversible neurodegeneration. Therefore, development has been made of therapeutic agents for Alzheimer’s disease, which are designed to utilize approaching strategies for targeting Aβ, such as inhibition against Aβ production and against Aβ-induced generation of reactive oxygen species or inflammation, removal of intracerebral Aβ by Aβ antibody, etc. Furthermore, abnormal changes in Aβ can be detected about 10-20 years before the onset of Alzheimer’s disease, indicating the possibility for early prevention of Alzheimer’s disease. In the past 20 years, many methods have been tried to lower toxic Aβ levels by inhibiting Aβ production and aggregation.

During the pathogenesis of Alzheimer’s disease, Aβ monomers are abnormally converted into oligomers and fibrils through oligomerization and fibrosis processes. Therefore, understanding this process is important for the discovery of drugs for Alzheimer’s pathogenesis. For example, the toxicity caused by Aβ oligomers can be reduced by inhibiting the nucleation phenomenon, which is a step for generating oligomers, but can also be exacerbated by inhibiting only the elongation step. The assay using thioflavin T, which specifically binds to Aβ fibrin, can be used as a tool for drug discovery because it can quickly and easily investigate the inhibitory effect of drugs on Aβ fibrosis. The recently developed Multimer Detection System (MDS), which is a technology capable of detecting only oligomeric proteins, has been reported to diagnose Alzheimer’s disease by measuring Aβ oligomerization in human blood. A strategy to simultaneously interpret drug-induced changes in nucleation and Aβ aggregation by combining ThT and MDS analysis will provide an opportunity to systematically discover therapeutic agents for Alzheimer’s disease.

In addition to the aggregation of abnormal proteins, neuroinflammation has begun to attract attention as another important factor in the course of onset and development of Alzheimer’s disease. When the central nervous system is damaged, white blood cells (leukocytes), microglia, and astrocytes are activated and various inflammation-related substances are secreted. This series of comprehensive reactions is defined as “neuroinflammatory response”.

The acute inflammatory response of the cranial nerve is a mechanism that protects the brain from direct and indirect damage caused by infections and toxic substances, but if the inflammation-related signals are imbalanced due to various factors, the stage may be shifted to a chronic inflammatory response. In other words, the initial acute inflammatory response has a protective action, but as the inflammatory response becomes chronic, it negatively affects nerve cells. This chronic neuroinflammatory state activates glia cells, such as microglia cells, and promotes the secretion of various cytokines therefrom, and nerve damage is continuously made due to these immune responses. Therefore, studies are being actively conducted on the role of inflammation-related factors such as microglia, astrocyte, and cytokine in the pathophysiology of Alzheimer’s disease and the development of therapeutic agents based thereon.

It has been reported that chemical substances are used for various diseases including neurological complications through experiments on Alzheimer’s disease. Among such chemical substances, isoflavones are one kind of the candidates for drug development in Alzheimer’s disease. Isoflavones are diphenol compounds widely present in the plant kingdom and include glycoside forms such as genistin, daidzin, glycitin, etc., and non-glycoside genistein, daidzein, glycitein, etc. Genistein and its derivatives found in soy proteins exhibit anti-angiogenic, anti-cancer, anti-inflammatory, anti-neuroinflammatory, and neuroprotective effects. In particular, genistein can exert a protective effect against neuronal cell degeneration by attenuating Aβ deposition, hyperphosphorylation, and neuroinflammation in mice. Similarly, equol, which is a bacterial metabolite of daidzein isoflavone, showed anticancer, anti-neuroinflammatory, and neuroprotective effects.

Leading to the present disclosure, intensive and thorough research conducted by the present inventors resulted in the finding that novel chromene derivatives derived from isoflavones are effective for treating degenerative brain diseases.

### DISCLOSURE OF INVENTION

### Technical Problem

The present disclosure aims to provide a novel isoflavone-derived chromene derivative compound effective for the prevention, treatment, or alleviation of a degenerative brain disease.

### Solution to Problem

To achieve the goal, an aspect of the present disclosure provides a pharmaceutical composition including the compound represented by Chemical Formula 1 described herein, a solvate thereof, a hydrate thereof, or a pharmaceutically acceptable salt thereof as an active ingredient for the prevention or treatment of a degenerative brain disease.

Another aspect of the present disclosure provides a health functional food composition including the compound, a solvate thereof, a hydrate thereof, or a pharmaceutically acceptable salt thereof for preventing or alleviating a degenerative brain disease

Another aspect of the present disclosure provides a method for the treatment of a degenerative brain disease, the method including a step of administering the compound, a solvate thereof, a hydrate thereof, or a pharmaceutically acceptable salt thereof to a subject in need thereof.

Another aspect of the present disclosure provides a use of the compound, a solvate thereof, a hydrate thereof, or a pharmaceutically acceptable salt thereof for preparing a medicament for the prevention or treatment of a degenerative brain disease.

### Advantageous Effects of Invention

The compound, a solvate thereof, a hydrate thereof, or a pharmaceutically acceptable salt thereof, provided according to an aspect of the present disclosure, has high inhibitor activity against Aβ fibrosis and oligomerization and exhibits antioxidant and anti-inflammatory actions to protect nerve cells as well as to improve cognitive function, thereby finding advantageous applications in the prevention, alleviation, or treatment of degenerative brain diseases, especially Alzheimer’s disease.

### BRIEF DESCRIPTION OF DRAWINGS

FIG. 1 is a plot of inhibitory effects on fibrosis of Aβ42 according to drugs.

FIG. 2 is a graph of inhibitory effects of drugs on oligomerization of Aβ42 as measured by MDS.

FIG. 3 is a graph showing cytotoxicity of drugs against BV2 cells.

FIG. 4 shows graphs of inhibitory effects of SPA1427 on cytotoxicity (upper) and nitric oxide production (lower) in LPS-treated BV2 cells.

FIG. 5 is a graph showing cytotoxicity of drugs against C6 cells.

FIG. 6 is a graph showing cytotoxicity of drugs against N2a cells.

FIG. 7 is a graph showing cytotoxicity of drugs against SH-SY5Y cells.

FIG. 8a is a graph of inhibitory effects of drugs on nitric oxide production in LPS-treated BV2 cells.

FIG. 8b is a graph showing cytotoxicity of drugs against LPS-treated BV2 cells.

FIG. 9a is a graph of inhibitory effects of drugs on nitric oxide production in LPS-treated primary microglia.

FIG. 9b is a graph showing cytotoxicity of drugs against LPS-treated primary microglia.

FIG. 10a is a graph showing inhibition results of drugs against IL-6 expression in LPS-treated BV2 cells.

FIG. 10b is a graph showing inhibition results of drugs against TNF-a expression in LPS-treated BV2 cells.

FIG. 11a is a graph showing inhibition results of drugs against COX-2 expression in LPS-treated BV2 cells.

FIG. 11b is a graph showing inhibition results of drugs against iNOS expression in LPS-treated BV2 cells.

FIG. 12a is a graph showing inhibition results of drugs against JNK phosphorylation in LPS-treated BV2 cells.

FIG. 12b is a graph showing inhibition results of drugs against p38 phosphorylation in LPS-treated BV2 cells.

FIG. 13 is a graph of nerve growth factor release measurements in C6 cells.

FIG. 14a is a graph of neurite outgrowth measurements in N2a cells.

FIG. 14b shows images for neurite outgrowth in N2a cells.

FIG. 15 is a graph of inhibition against beta amyloid oligomers in primary microglia.

FIG. 16 shows graphs of cytotoxicity against LPS-treated HEK293 cells and HEK293 cells overexpressing the dementia gene PSEN1.

FIG. 17 shows graphs of inhibitory activity of the compound of the present disclosure against PSEN1 expression in HEK293 cells and HEK293 cells overexpressing the dementia gene PSEN1.

FIG. 18 is a graph showing results of breaking MGO-AGEs according to concentrations of SPA1413.

FIG. 19 is a graph showing results of breaking MGO-AGEs upon treatment with the compounds of the present disclosure.

FIGS. 20 (a to e) show graphs of effects on object recognition ability and spatial memory after the compounds of the present disclosure are administered to 5xFAD mice.

FIG. 21 (a and b) show graphs of effects on alteration behavior after the compounds of the present disclosure are administered to 5xFAD mice.

FIG. 22 is a graph of effects on avoidance memory recovery after the compounds of the present disclosure are administered to 5xFAD mice.

FIG. 23 shows fluorescence microphotographic images of stained mouse tissues used in a cognitive function improvement test.

FIGS. 24 and 25 are graphs comparing numbers of amyloid plaques in stained mouse tissues used in cognitive function improvement tests.

FIG. 26 shows images of microglia positive to MHC II in the mouse cortex and hippocampus.

FIG. 27 shows graphs of numbers of microglia positive to MHC II in the mouse cortex and hippocampus.

FIG. 28 shows expression of Oligomeric Aβ proteins in the mouse cortex and hippocampus.

FIG. 29 is a plot of body weight changes with time.

### BEST MODE FOR CARRYING OUT THE INVENTION

Below, a detailed description will be given of the present disclosure.

The present disclosure provides a pharmaceutical composition including a compound represented by Chemical Formula 1, a solvate thereof, a hydrate thereof, or a pharmaceutically acceptable salt thereof as an active ingredient for preventing or treating a degenerative brain disease:

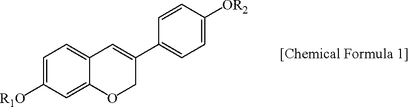

wherein,
R₁ and R₂ are each independently a hydrogen atom, or
a linear or branched alkyl carbonyl of C1-10.

In an embodiment,
R₁ and R₂ may each be independently a hydrogen atom, or
a linear or branched alkyl carbonyl of C2-8.

In another embodiment,
R₁ and R₂ may each be independently a hydrogen atom,

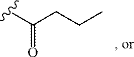

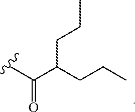

In another embodiment, [0063] the compound represented by Chemical Formula 1 may be any one selected from the group consisting of:
(1) 3-(4-hydroxyphenyl)-2H-chromen-7-ol;
(2) 4-(7-(butyryloxy)-2H-chromen-3-yl)phenyl butyrate);
(3) 4-(7-((2-ethylpentanoyl)oxy)-2H-chromen-3-yl)phenyl 2-ethylpentanoate.

The compound, represented by Chemical Formula 1, of the present disclosure may be used in a form of a pharmaceutically acceptable salt, with preference for an acid addition salt formed with a pharmaceutically acceptable free acid. Acid addition salts may be obtained from inorganic acids such as hydrochloric acid, nitric acid, phosphoric acid, sulfuric acid, hydrobromic acid, hydroiodic acid, nitrous acid, phosphorous acid, etc., and organic acids such as aliphatic mono- and dicarboxylates, phenyl-substituted alkanoates, hydroxy alkanoates, alkanedioates, aromatic acids, aliphatic and aromatic sulfonic acids, trifluoroacetic acid, acetate, benzoic acid, citric acid, lactic acid, maleic acid, gluconic acid, methanesulfonic acid, 4-toluenesulfonic acid, tartaric acid, fumaric acid and the like. Examples of such pharmaceutically non-toxic salts include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartrate, methanesulfonate, propanesulfonate, naphthalene-1-sulfonate, naphthalene-2-sulfonate, mandelate, and the like.

The acid addition salt according to the present disclosure can be prepared by a conventional method. For example, the derivative of Chemical Formula 1 is dissolved in an organic solvent, such as methanol, ethanol, acetone, methylene chloride, acetonitrile, etc., and added with an organic or inorganic acid to form a precipitate which is then filtered and dried. Alternatively, crystallization in the organic solvent can be conducted by drying the solvent and excess acid through vacuum distillation.

Furthermore, the present disclosure includes not only the compound represented by Formula 1 and pharmaceutically acceptable salts thereof, but also solvates, optical isomers, hydrates, and the like, which can be prepared therefrom.

As used herein, the term “hydrate” refers to a compound of the present disclosure or a salt thereof that bears a stoichiometric or non-stoichiometric amount of water bound by non-covalent intermolecular forces or salts thereof. A hydrate of the compound, represented by Chemical Formula 1, of the present disclosure may contain a stoichiometric or non-stoichiometric amount of water that is bound by non-covalent intermolecular forces. The hydrate may contain water in an amount of one or more equivalents and preferably in an amount of one to five equivalents. Such a hydrate may be prepared by crystallizing a compound, represented by Formula 1, of the present disclosure, an isomer thereof, or a pharmaceutically acceptable salt thereof from water or a solvent containing water.

As used herein, the term “solvate” means a compound of the present disclosure or a salt thereof which bears either a stoichiometric or non-stoichiometric amount of a solvent bound by non-covalent intermolecular forces. In this regard, preferred are solvents that are volatile, non-toxic, and/or suitable for administration to humans.

The term “isomer”, as used herein, refers to a compound of the present disclosure or a salt thereof that has the same chemical or molecular formula, but differs structurally or sterically. Such isomers include structural isomers such as tautomers, stereoisomers such as R or S isomers having an asymmetric carbon center, geometric isomers (trans, cis), and optical isomers (enantiomers). All these isomers and racemates thereof also fall within the scope of the present disclosure.

The present disclosure claims a method for preparing a compound represented by Formula 1, a solvate thereof, a hydrate thereof, or a pharmaceutically acceptable salt thereof.

### Preparation Example

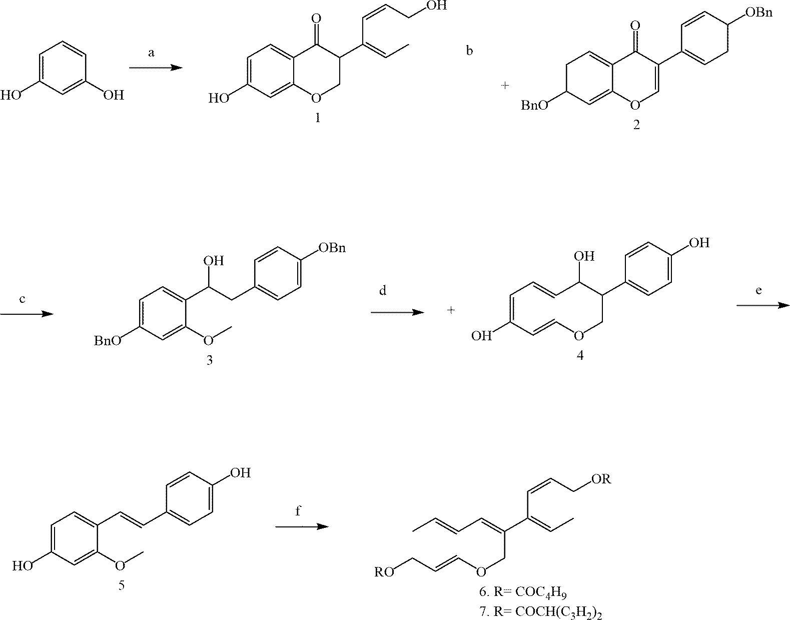

Reaction conditions: (a) i) (4-hydroxyphenyl) acetic acid, BF_{3.}Et₂O, 120° C. 10 min; ii) DMF, 50° C.; iii) MeSO₂Cl, 80° C.(b) BnBr, K₂CO₃, DMF, 40° C. 2 h; (c) NaBH₄, THF, EtOH, rt, 4 h; (d) 20% Pd(OH)₂, Ammonium formate, EtOH, THF, H₂O, 100° C. 35 min; (e) 20% HCl-EtOH, EtOH, rt, 45 min; (f) butyryl chloride or 2-propylpentanoyl chloride, pyridine, DCM, rt, 4 h.

The present disclosure provides a pharmaceutical composition including a compound represented by Formula 1, a solvate thereof, a hydrate thereof, or a pharmaceutically acceptable salt thereof as an active ingredient for the prevention or treatment of Alzheimer’s disease.

The degenerative brain disease may be any one selected from the group consisting of Parkinson’s disease, Alzheimer’s disease, Huntington’s disease, alcoholic cranial nerve disease, spinal cord injury, alcoholic dementia, and Wernicke-Korsakoff’s syndrome.

The compound of the present disclosure inhibits fibrosis and oligomerization of Aβ42. In this regard, the compound inhibits irreversible neurodegeneration by suppressing Aβ-induced generation of reactive oxygen species or inflammation or removing intracerebral Aβ with an Aβ antibody, thereby allowing for prophylaxis, palliation, and therapy of Alzheimer’s disease.

Aβ is produced as amyloid precursor protein (APP) is decomposed by β and γ-secretases. This abnormal accumulation of Aβ is considered to be a cause of the neuronal degeneration associated with AD. Aβ monomers are aberrantly converted to oligomers and fibrin by oligomerization and fibrillation processes during the pathogenesis of Alzheimer’s disease. Aβ-induced toxicity mechanism is deeply related to Aβ fibrosis, and during this fibrosis process, various types of aggregates, such as soluble oligomers, protofibrils, and amyloid fibrils, are formed.

In addition to the aggregation of abnormal proteins neuroinflammation is another important factor involved in the onset and development of Alzheimer’s disease. When the central nervous system is damaged, leukocytes, microglia, and astrocytes are activated to secrete various inflammation-related substances. It is known that an increase in the inflammatory response can cause Alzheimer’s disease by increasing the accumulation of beta-amyloid as well as damage to nerve cells.

Nerve growth factor is an important protein for the growth, differentiation, survival, and maintenance of function of a specific neuron, and promotes the function of a nerve cell. Insufficiency of the nerve growth factor with such functions leads to death of the cells. In the pathological development of Alzheimer’s disease, the nerve growth factor system undergoes a change. This change brings about the secondary effects of degrading the function of cholinergic neurons, reducing the plasticity of brain neurons, promoting the progression of Alzheimer’s disease such as amyloid protein deposition and tau protein hyperphosphorylation. As a result, neurodegenerative signals become dominant, leading to the onset of Alzheimer’s disease and the declination of cognitive functions.

Presenilin-1 (PSEN1) is a gene associated with familial Alzheimer’s disease. Some mutations in this gene can give rise to the production of more toxic Aβ fragments. The protein encoded by PSEN1 accounts for an active domain of γ-secretase, and this protein complex is involved in cleavage of the Notch receptor. PSEN1 is also involved in the release of synaptic transmitters in hippocampal vertebral neuritis. In addition, mutations in PSEN1 are associated with Alzheimer’s disease, and some of the mutations also closely correlate with other neurological diseases such as frontotemporal dementia and ALS.

Advanced glycoxidation end-products are substances such as proteins that become glycated. They may be produced in baked or fried foods or as a result of combination between proteins and sugars in the body. Advanced glycation end-products, which are undegradable waste products, may generate cytotoxic reactive oxygen species, causing diabetes, cancer, arteriosclerosis, hypertension, and hyperlipidemia. Damage by such substances on cranial nerves is known to provoke Alzheimer’s disease.

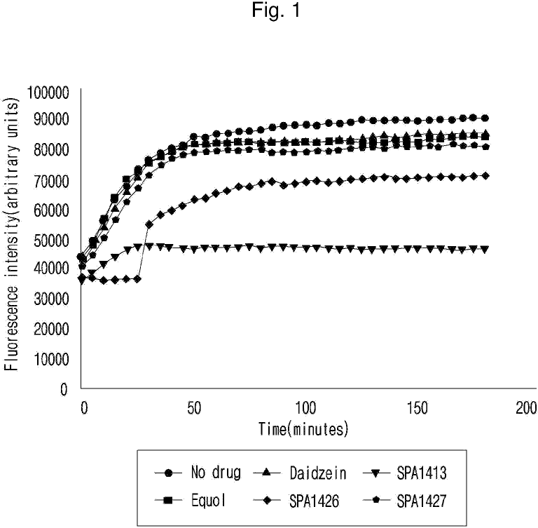

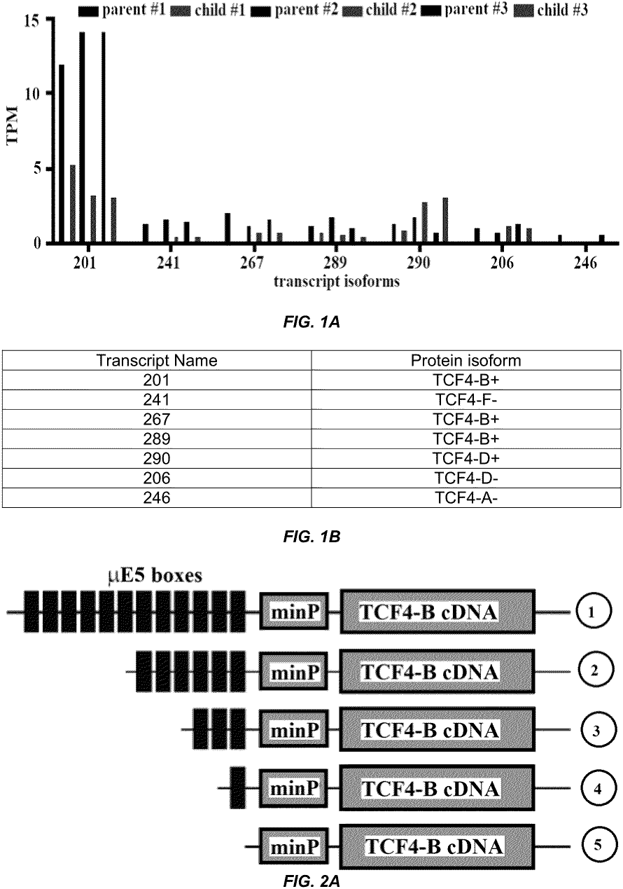

In a specific example of the present disclosure, it was confirmed that the compounds SPA1413, SPA1426, and SPA1427 of the present disclosure exhibit the effect of inhibiting Aβ42 fibrosis (FIG. 1) and Aβ42 oligomerization (FIG. 2).

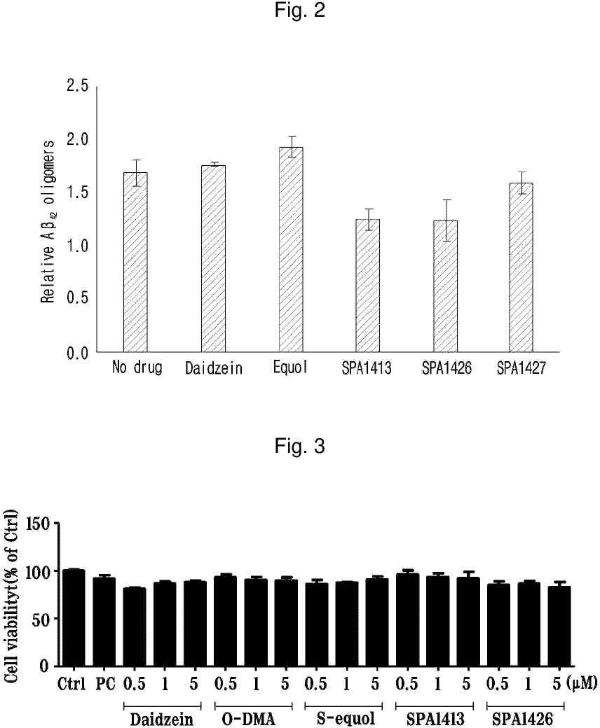

When mice-derived microglia (BV2 cells) and neurons (N2a cells), rat-derived glial cells (C6 cells), and human-derived neurons (N2a cells) were treated with the compounds SPA1413, SPA1426, and SPA1427 of the present disclosure, no cytotoxicity was detected. The compounds of the present disclosure were observed to suppress the LPS-induced overproduction of nitric oxide in BV2 cells (FIGS. 3 to 9).

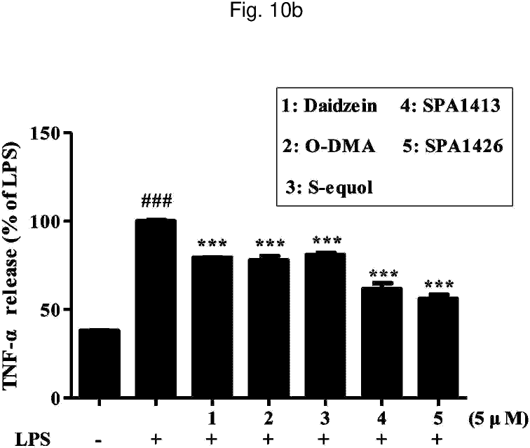

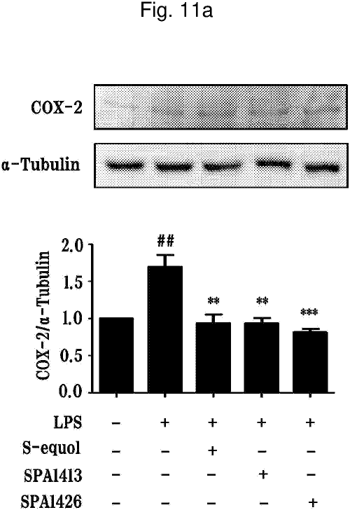

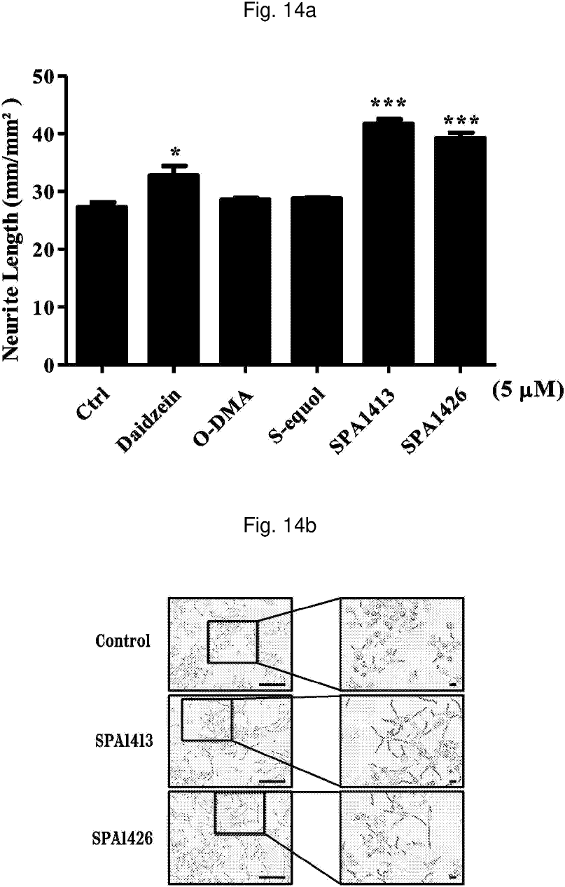

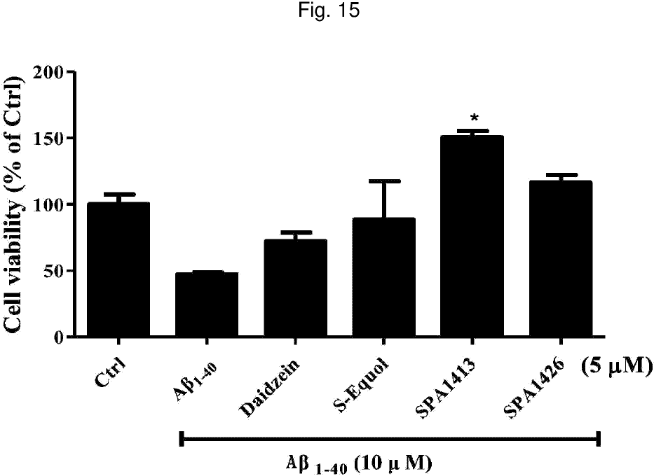

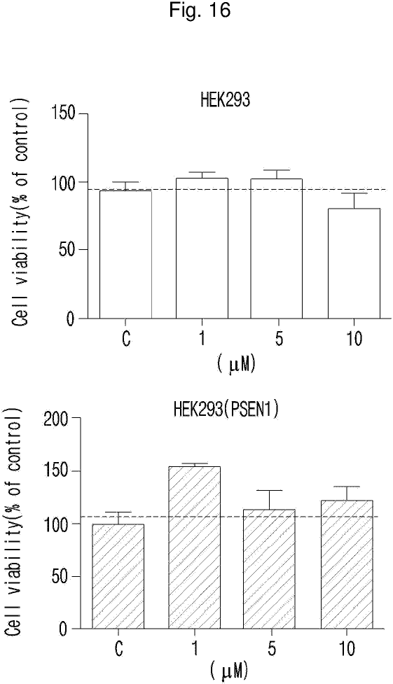

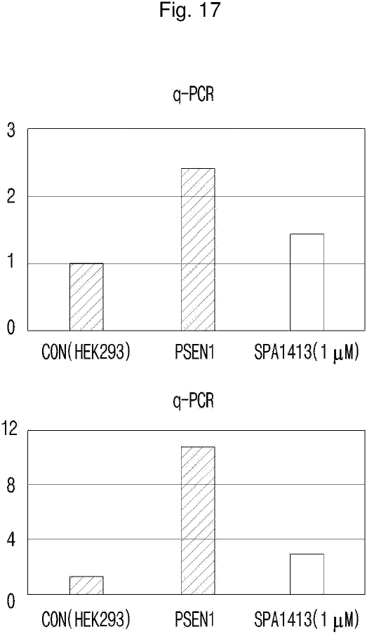

In addition, the inflammatory factors (IL-6, TNF-a, COX-2, iNOS, JNK, and p38) that are overexpressed by LPS treatment in BV2 cells decreased in expression level with the application with the compounds of the present disclosure thereto (FIGS. 10 to 12). Treatment with the compounds of the present invention was observed to increase the secretion of nerve growth factor in C6 cells (FIG. 13) and neurite outgrowth in N2a cells (FIG. 14). Also, the cell viability of primary microglial cells decreased by treatment with Aβ₁₋₄₀, but increased by treatment of the compounds of the present disclosure (FIG. 15).

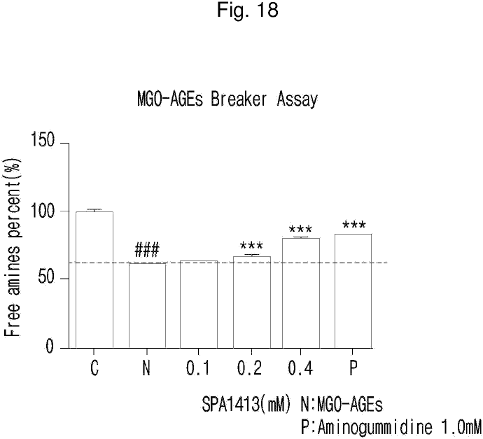

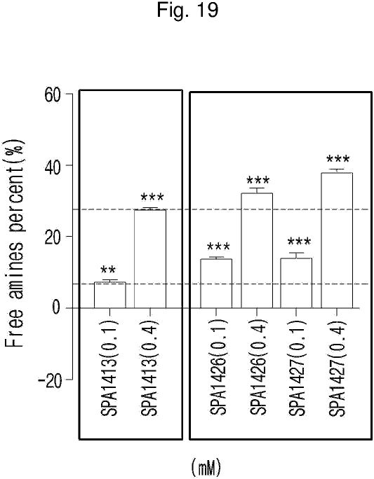

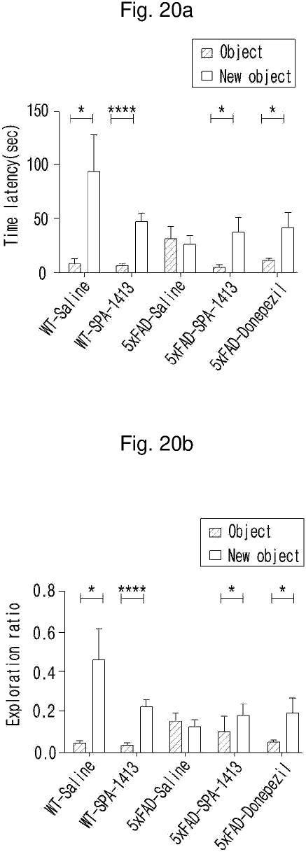

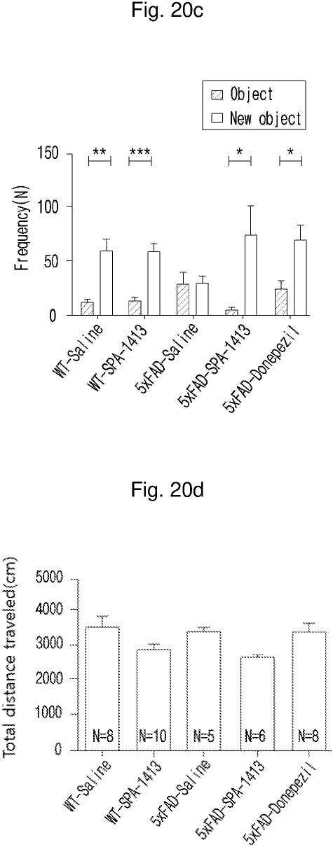

In a specific example, when applied to PSEN1-overexpressed HEK293 cells, the compounds of the present disclosure inhibited the expression of PSEN1 (FIGS. 16 and 17), and were found to have an ability to break down the advanced glycation end-products (FIGS. 18 and 19)

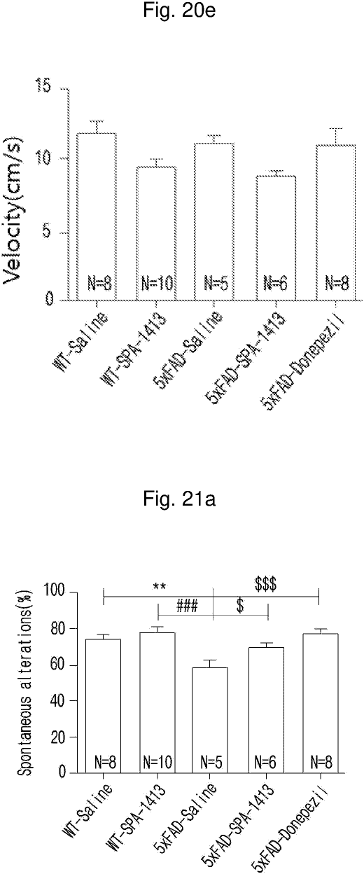

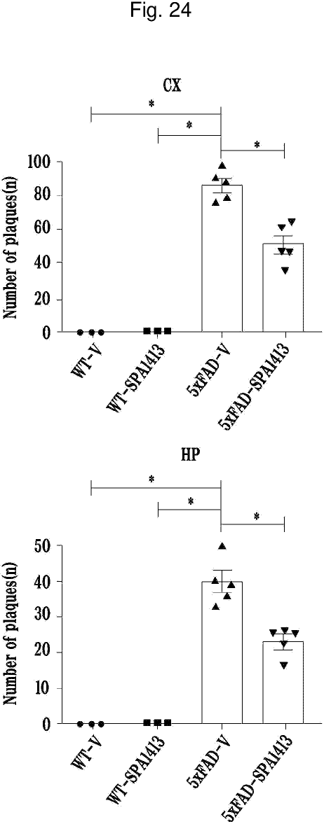

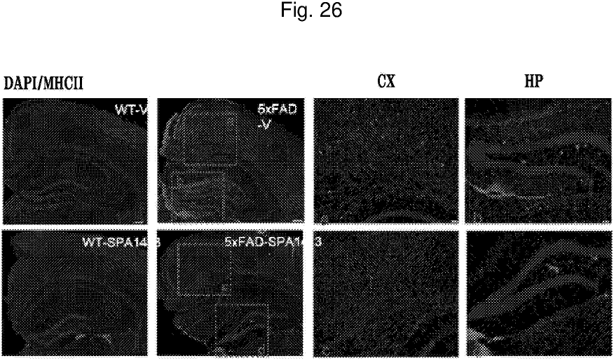

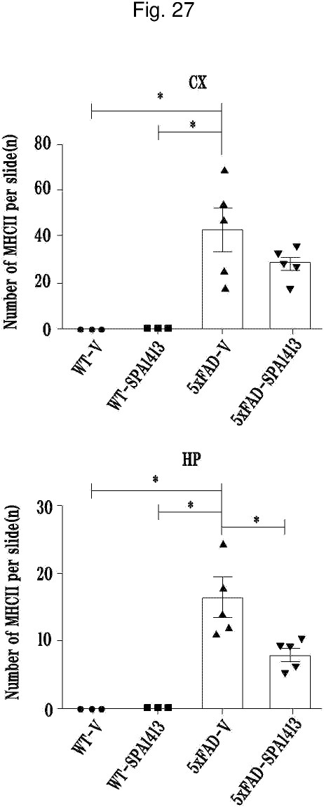

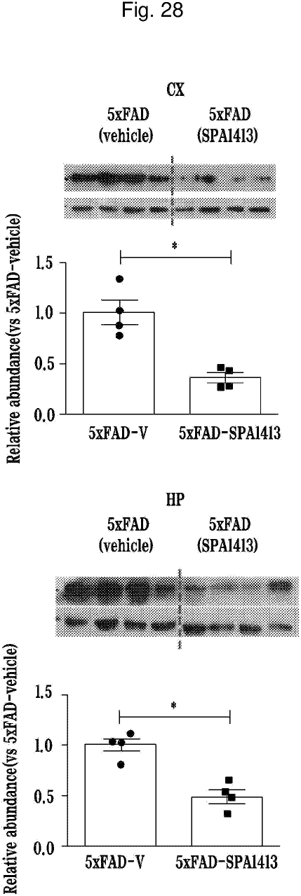

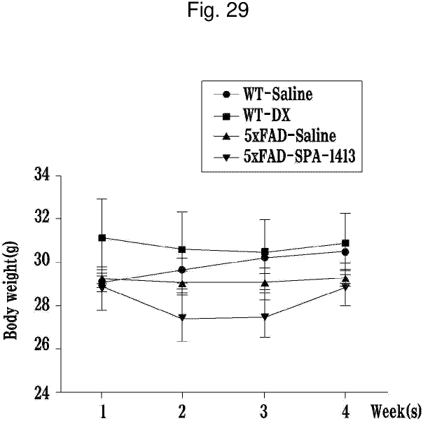

In an in-vivo assay using an animal model of dementia to which the compounds of the present invention were administered, the animals did not change in body weight (FIG. 29), but exhibited an improvement in object recognition ability, spatial memory ability, alteration behavior, and avoidance memory recovery (FIGS. 20 to 22). Stained tissues of mice to which the compounds of the present disclosure were administered were observed to decrease the number of amyloid plaques (FIGS. 23 to 25) and the number of MHC II-positive microglial cells (FIGS. 26 and 27) and decrease the expression of oligomeric Aβ proteins (FIG. 28), indicating that the compounds of the present disclosure can be advantageously used as a pharmaceutical composition for the prevention or treatment of Alzheimer’s disease.

The compound represented by Chemical Formula 1 or a pharmaceutically acceptable salt thereof may be administered in the form of various oral and parenteral formulations during clinical administration. When formulated into dosage forms, use may be made of common diluents or excipients such as fillers, extenders, binders, humectants, disintegrants, surfactants, etc. Solid formulations for oral administration include tablets, pills, powders, granules, capsules, etc. Such solid formulations are prepared by mixing at least one of the compounds with at least one excipient, e.g., starch, calcium carbonate, sucrose or lactose, gelatin, etc. In addition to simple excipients, lubricants such as magnesium stearate and talc are also used. Liquid formulations for oral administration include suspensions, internal solutions, emulsions, syrups, etc. In addition to commonly used simple diluents such as water and liquid paraffin, the formulation may contain various excipients such as humectants, sweeteners, fragrances, and preservatives. Formulations for parenteral administration include sterile aqueous solutions, non-aqueous solutions, suspensions, and emulsions. Non-aqueous solvents and suspensions may include propylene glycol, polyethylene glycol, vegetable oils such as olive oil, and injectable esters such as ethyl oleate.

A pharmaceutical composition including the compound represented by Formula 1 or a pharmaceutically acceptable salt thereof as an active ingredient may be administered orally or parenterally, and parenteral administration may be accounted for by injection via subcutaneous, intravenous, intramuscular, or intrathoracic routes.

The effective dosage of the pharmaceutical composition varies depending on patient’s weight, age, gender, health condition, diet, administration frequency, administration method, excretion and severity of disease, but these cannot limit the present disclosure by any means. An individual dosage preferably contains the amount of active compound that is suitable for being administered in a single dose.

Another aspect of the present disclosure provides a health functional food composition including the compound represented by Formula 1, a solvate thereof, a hydrate thereof, or a pharmaceutically acceptable salt thereof as an active ingredient for preventing or alleviating Alzheimer’s disease.

The present disclosure provides a method for treating Alzheimer’s disease, the method including a step of administering the compound to a subject in need thereof. Further, another aspect of the present disclosure provides the compound for use in the prevention or treatment of Alzheimer’s disease. Furthermore, another aspect of the present invention provides a use of the compound for preparing a medicament for the prevention or treatment of Alzheimer’s disease.

Hereinafter, the present disclosure will be described in detail through Examples and Experimental Examples.

However, the Examples and Experimental Examples described below are merely illustrative of the present disclosure in detail in one aspect, and the present disclosure is not limited thereto.

### MODE FOR CARRYING OUT THE INVENTION

### <Preparation Example 1> Preparation of 7-Hydroxy-3-(4-hydroxyphenyl)chromen-4-one

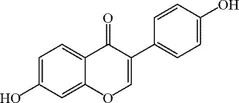 [SMILES: O=c1c(-c2ccc(O)cc2)coc2cc(O)ccc12]

In a two-neck round-bottom flask, resorcinol (2.0 g, 18.16 mmol) and 4-hydroxyphenylacetic acid (2.8 g, 18.16 mmol) were purged with nitrogen gas and then slowly added with drops of boron trifluoro etherate (5.2 mL) over 10 minutes at 120° C. while being stirred. The temperature was lowered to room temperature to form a white solid which was then completely dissolved in DMF (24 mL) and stirred at 50° C. for 10 minutes. After temperature elevation to 80° C., methane sulfonyl chloride (13.7 g, 119.88 mmol) was added to conduct the reaction for 30 minutes. When the reaction progressed to completion as monitored by TLC, the reaction was terminated by adding water of 0° C. Following extraction with ethyl acetate, the organic layer thus formed was washed with NaHCOs, water, and saturated brine, dried over MgSO₄, filtered, and concentrated in a vacuum. The concentrate was washed with chloroform, water, and a small amount of MeOH to afford Compound 1 as an ivory solid (1.9 g, 7.56 mmol, 41.6%).

Rf= 0.28 (n-hexane/EtOAc = 1:1); ¹H NMR (400 MHz, DMSO) δ(OH, s), 9.55 (OH, s), 8.29 (1H, s), 7.96 (1H, d, J= 8.8 Hz), 7.38 (2H, d, J= 8.8 Hz), 6.93 (1H, dd, J= 8.8, 2.0 Hz), 6.85~6.79 (3H, m); ¹³C NMR (100 MHz, DMSO) δ172.1, 166.9, 166.7, 162.3, 139.6, 136.8, 132.9, 132.0, 124.6, 124.4, 114.1, 111.6.

### <Preparation Example 2> Preparation of 7-Benzyloxy-3-(4-benzyloxyphenyl)chromen-4-one

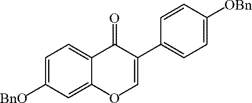 [SMILES: O=c1c(-c2ccc(OCc3ccccc3)cc2)coc2cc(OCc3ccccc3)ccc12]

Compound 1 (540.8 mg, 2.11 mmol) was dissolved in dimethylformamide (2 mL) and added with K₂CO₃ (961.8 mg, 6.38 mmol) before being stirred for 30 minutes. After addition of benzyl bromide (1.1 g, 6.38 mmol), the reaction was conducted at 40° C. for 2 hours. When the reaction progressed to completion as monitored by TLC, the reaction was terminated with water. Following extraction with ethyl acetate, the organic layer thus formed was washed with water, and saturated brine, dried over MgSO₄, filtered, and concentrated under reduced pressure. The concentrate was purified by column chromatography to afford Compound 2 as a white solid (743.2 mg, 1.70 mmol, 79%).

R _{f}= 0.27 (n-hexane/EtOAc = 4:1); ¹H NMR (400 MHz, CDCl ₃) δ J = 9.2 Hz), 7.91 (1H, s), 7.50~7.33 (10H, m), 7.07~7.03 (3H, m), 6.93 (1H, s), 5.18 (2H, s), 5.11 (2H, s); ¹³C NMR (100 MHz, CDCl ₃) δ.9, 152.1, 135.7, 130.1, 128.8, 128.6, 128.4, 127.9, 127.5, 127.4, 115.0, 114.9, 105.0, 101.3, 70.5, 70.1

### <Preparation Example 3> Preparation of 7-Benzyloxy-3-(4-benzyloxyphenyl)chroman-4-ol

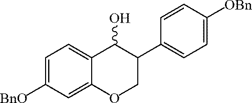 [SMILES: OC1c2ccc(OCc3ccccc3)cc2OCC1c1ccc(OCc2ccccc2)cc1]

Compound 2 (743.2 mg, 1.70 mmol) was completely dissolved in THF-EtOH (10:1 v/v, 22 mL) and added with NaBH₄ (302.6 mg, 8.21 mmol). The reaction was conducted at room temperature for 24 hours. When the reaction progressed to completion as monitored by TLC, the reaction was terminated with water, followed by extraction with ethyl acetate. The organic layer thus formed was washed with water and saturated brine, dried over MgSO₄, filtered, and concentrated under reduced pressure. The residue thus obtained was purified by column chromatography to afford Compound 3 as a white solid (410 mg, 1.44 mmol, 48%).

R f= 0.30 (chloroform/ n-hexane/ethyl acetate = 20:5:1); ¹HNMR (400 MHz, CDCl ₃) δ(5H, m), 7.21 (3H, d, J = 8.8 Hz), 7.12 (1H, d, J = 8.4 Hz), 6.82 (2H, d, J = 8.8 Hz), 6.51 (1H, dd, J = 8.4, 2.4 Hz), 4.57 (1H, d, J = 4.8 Hz), 4.58 (2H, s), 4.49 (1H, dd, J = 11.2, 3.2 Hz), 4.34 (1H, dd, J = 11.2, 4.8 Hz), 3.78 (3H, s), 3.76 (3H, s), 3.34 (1H, m); ¹³CNMR (100 MHz, CDCl₃) δ154.1, 152.9, 143.3, 139.2, 124.5, 121.1, 120.0, 109.4, 102.8, 101.1, 100.7, 95.3, 70.6, 71.0, 39.3.

### <Preparation Example 4> Synthesis of 3-(4-Hydroxyphenyl)chroman-4,7-diol

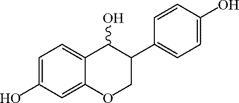 [SMILES: Oc1ccc(C2COc3cc(O)ccc3C2O)cc1]

Compound 3 (100 mg, 0.21 mmol) was dissolved in EtOH-THF (2:1 v/v, 15 mL), added with 20% Pd(OH)₂ (100 mg, 100 wt%), and then heated to 120° C. while being stirred. After the temperature was lowered to room temperature, a 1 M ammonium formate solution (248.9 mg, 3.9 mmol) was dropwise added. The mixture was heated to 120° C. and reacted for 1 hour. When the reaction was completed as monitored by TLC, the reaction mixture was filtered through a celite pad, washed with methanol, and concentrated in a vacuum. Purification by column chromatography afforded Compound 4 as a white solid (30.7 mg, 0.09 mmol, 45%).

R f = 0.18 (n-hexane/ethyl acetate =1/1); ¹H NMR (400 MHz, CD ₃OD) δ(1H,d, J = 8.4 Hz), 7.03 (2H, d, J= 8.8 Hz), 6.71 (2H, d, J= 8.8 Hz), 6.38 (1H; dd; J= 8.4, 2.4 Hz), 6.25 (1H, d, 2.4 Hz), 4.74 (1H, d, J = 2.4 Hz), 4.24 (1H, dd, J = 11.6, 3.2 Hz), 4.14 (1H; dd; J = 10.0, 8.8 Hz), 3.01~2.96 (1H, m); ¹³C NMR (100 MHz, CD ₃OD) δ157.4, 156.8, 131.9, 130.9, 130.1, 117.9, 116.3, 109.6, 103.3, 69.9, 69.3, 47.7.

### <Example 1> Synthesis of 3-(4-Hydroxyphenyl)-2H-chromen-7-ol (SPA1413)

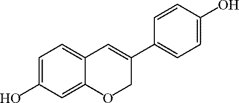 [SMILES: Oc1ccc(C2=Cc3ccc(O)cc3OC2)cc1]

Compound 4 (50 mg, 0.19 mmol) was dissolved in EtOH (2 mL) and added with 20% HCl-EtOH (0.2 mL) before being stirred for 45 minutes. When the reaction progressed to completion as monitored by TLC, the reaction was terminated with water. Following extraction with ethyl acetate, the organic layer thus formed was washed with water, and saturated brine, dried over MgSO₄, filtered, and concentrated under reduced pressure. The concentrate was purified by column chromatography to afford Compound 5 as a reddish brown solid (23 mg, 0.09 mmol, 49%).

R _{f} = 0.24 (n-hexane/EtOAc = 2:1); ¹H NMR (400 MHz, CD ₃OD) δ7.29 (2H, d, J = 6.8 Hz), 6.89 (1H, d, J = 6.4 Hz), 6.78 (2H, d, J = 6.8 Hz), 6.67 (1H, s), 6.34 (1H; dd, J = 6.4, 1.6 Hz), 6.26 (1H, d, J = 1.6 Hz), 5.02 (2H, S); ¹³C NMR(100 MHz, CD₃OD)δ158.3, 155.7, 129.8, 129.4, 128.6, 126.8, 118.5, 116.9, 116.5, 105.6, 103.5, 68.1.

### <Example 2> Synthesis of 4-(7-(butyryloxy)-2H-chromen-3-yl)phenyl butyrate (SPA1426)

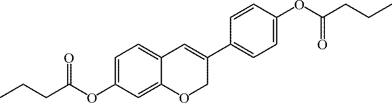 [SMILES: CCCC(=O)Oc1ccc(C2=Cc3ccc(OC(=O)CCC)cc3OC2)cc1]

Compound 5 (200 mg, 0.83 mmol) was dissolved in anhydrous dichloromethane (DCM, 5 ml) and then added with butyryl chloride (0.5 ml, 4.8 mmol) and pyridine (0.5 ml, 6 mmol) before being stirred at room temperature for 4 hours. When the reaction progressed to completion as monitored by TLC, the reaction was terminated. The solvent was removed by concentration under reduced pressure. The residue thus obtained was dissolved in ethyl acetate and the organic layer was washed with water, and saturated brine, dried over NaSO₄, filtered, and concentrated in a vacuum. The concentrate was purified by column chromatography to afford Compound 6 (160 mg, 0.42 mmol, Yield = 50%).

R _{f} = 0.50 (n-hexane/ethyl acetate = 90:10); ¹H NMR (400 MHz, CH ₃OD) δd, J= 8.0 Hz), 7.02-7.05 (3H, m), 6.81 (s, 1H), 6.54 (1H, d, J= 8.0 Hz), 6.47 (s, 1H), 5.06 (s, 2H), 2.43-2.49(m, 4H), 1.63-1.69(m, 8H), 0.95 (6H, t, J= 8.0 Hz)

### <Example 3> Synthesis of 4-(7-((2-ethylpentanoyl)oxy)-2H-chromen-3-yl)phenyl 2-ethylpentanoate(SPA1427)

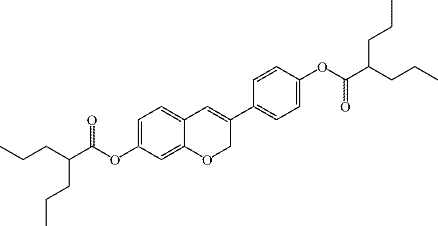 [SMILES: CCCC(CCC)C(=O)Oc1ccc(C2=Cc3ccc(OC(=O)C(CCC)CCC)cc3OC2)cc1]

Compound 5 (200 mg, 0.83 mmol) was dissolved in anhydrous dichloromethane (DCM, 5 ml) and then added with 2-propylpentanoyl chloride (0.5 ml, 3 millimoles) and pyridine (0.5 ml, 6 mmol) before being stirred at room temperature for 4 hours. When the reaction progressed to completion as monitored by TLC, the reaction was terminated. The solvent was removed by concentration under reduced pressure. The residue thus obtained was dissolved in ethyl acetate and the organic layer was washed with water, and saturated brine, dried over NaSO₄, filtered, and concentrated in a vacuum. The concentrate was purified by column chromatography to afford Compound 7 (150 mg, 0.3 mmol, Yield = 36%).

R _{f} = 0.80 (n-hexane/ethyl acetate = 95:5); ¹H NMR (400 MHz, CH ₃OD) δ7.56 (2H, d, J= 8.0 Hz), 7.10-7.17 (3H, m), 6.93 (s, 1H), 6.62 (1H, d, J= 8.0 Hz), 6.54 (s, 1H), 5.18 (s, 2H), 2.61-2.69 (m, 2H), 1.61-1.78 (m, 8H), 1.49-1.56 (m, 8H), 1.01 (12H, t, J= 8.0 Hz)

### <Experimental Example 1> Evaluation of Inhibitory Effect of Drugs on Aβ Oligomerization and Fibrosis

### <1-1> Thioflavin T (ThT) Assay

ThT assay was used to examine whether the drugs have an inhibitory activity against the fibrosis of Aβ. Lyophilized AggreSure AβAS-72216, AnaSpec) was dissolved in cold Tris buffered saline (TBS; pH 7.2) to prepare a 160 µg/mL Aβ solution. The solution was aliquoted into 1.5 mL tubes and stored at -80° C. until use. ThT (# T3516, Sigma-Aldrich) and drug candidates were dissolved in TBS to form a final concentration of 500 µM. The diluted aggregates were added to 386-well black plates (Nunc # 242764), mixed with ThT and drug candidates, and incubated at 37° C. for 180 minutes. Fluorescence signals were measured every 5 minutes using a plate reader (Victor3, PerkinElmer).

As can be seen from FIG. 1, the compounds (SPA1413, SPA1426, SPA1427) of the present disclosure showed the effect of inhibiting Aβ42 fibrosis, compared to the negative control (when only Aβ42 dilution buffer was added) and the isoflavone derivative compounds Daidzein or Equol.

### <1-2> MDS Assay

The drugs selected on the basis of the ThT assay results were evaluated for inhibitory activity against the oligomerization of Aβ42, using a multimer detection system (MDS) assay. Immediately after being dissolved in 1% NH₄OH (# 221228, Sigma-Aldrich), 1,1,1,3,3,3-hexafluoro-2-propanol-treated Aβ42 (# A-1163-1, rPeptide) was diluted in 500 µL of phosphate-buffered saline (PBS; pH 7.5), aliquoted into 1.5-mL tubes, and stored at -80° C. until use. Aβ42 and the compounds were mixed with PBS to make final concentrations of 200 µg/mL and 100 µM, respectively, and incubated at room temperature. Aggregation of Aβ42 was terminated by maintaining at -80° C.

After the reaction between each drug and Aβ42, the oligomeric form of Aβ42 was measured for each time to measure inhibitory effects on the oligomer. As can be seen in FIG. 2, the compounds of the present disclosure (SPA1413, SPA1426, and SPA1427) exhibited inhibitory effects on Aβ42 oligomerization, compared to the negative control (when only Aβ42 dilution buffer was added) and isoflavone derivative compounds Daidzein or Equol.

### <Experimental Example 2> Cytotoxicity Assay

### <2-1> Evaluation of Cytotoxicity in Mouse-Derived Microglia

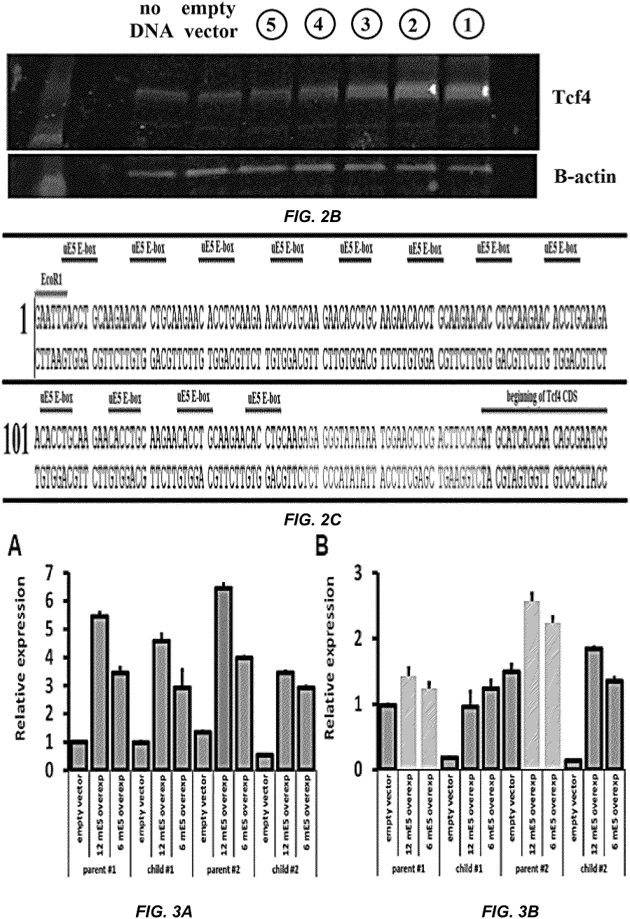

Mouse-derived microglia (BV2 cells) purchased from the Korean Cell Line Bank were seeded at a density of 4x10⁵ cells/well into 96-well plates, each well containing DMEM (Dulbecco’s Modified Eagle’s Media) supplemented with 10% FBS and 1% antibiotics, and stabilized by incubation for 24 hours. Thereafter, the cells were treated for 24 hours with various concentrations (0.5, 1, and 5 µM) of daidzein, o-desmethylangolensin (O-DMA), S-equal, SPA1413, and SPA1426. After removal of the medium, the cells were treated 0.5 mg/ml 3-(4,5-dimethylthiazol-2-yl)-2,5-diphenyltetrazolium bromide (MTT) solution 1 hour. The reduced formazan was dissolved in 150 µl of dimethyl sulfoxide (DMSO) and absorbance was read at 570 nm in a microspectrophotometer to determine cell viability. Cell viability of the cells treated with each sample was determined relative to that of the untreated normal control (Control), which was set to be 100%, and the results are depicted in FIGS. 3 and 4 (upper).

As can be seen from FIGS. 3 and 4 (upper), each sample was found to exhibit no toxicity when applied at various concentrations.

### <2-2> Evaluation of Cytotoxicity in Rat-Derived Neuroglia

Rat-derived microglia (C6 cells) purchased from the Korean Cell Line Bank were seeded at a density of 4x10⁵ cells/well into 96-well plates, each well containing DMEM (Dulbecco’s Modified Eagle’s Media) supplemented with 10% FBS and 1% antibiotics, and stabilized by incubation for 24 hours. Thereafter, the cells were treated for 24 hours with various concentrations (1 and 5 µM) of daidzein, o-desmethylangolensin, S-equal, SPA1413, and SPA1426.

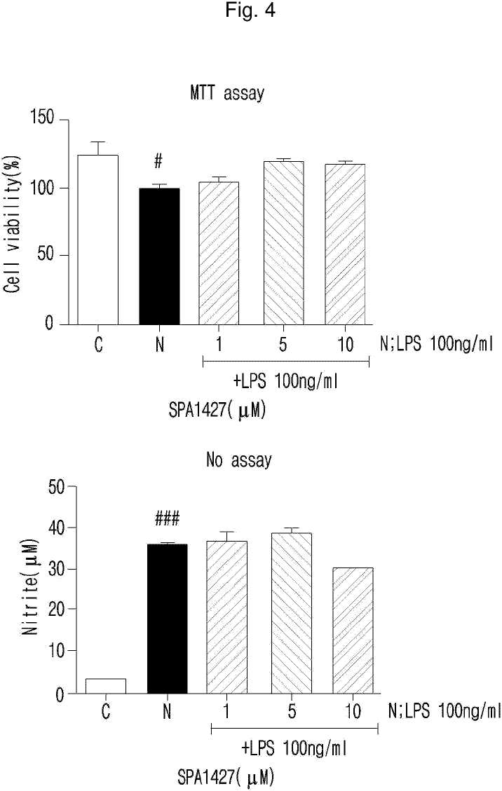

As can be seen from FIG. 5, each sample was found to exhibit no toxicity when applied at various concentrations.

### <2-3> Evaluation of Cytotoxicity in Mouse-Derived Neural Cells

Mouse-derived neural cells (N2a cells) purchased from the Korean Cell Line Bank were seeded at a density of 2x10⁵ cells/well into 96-well plates, each well containing DMEM (Dulbecco’s Modified Eagle’s Media) supplemented with 10% FBS and 1% antibiotics, and stabilized by incubation for 24 hours. Thereafter, the cells were treated for 24 hours with various concentrations (1 and 5 µM) of daidzein, o-desmethylangolensin, S-equal, SPA1413, and SPA1426.

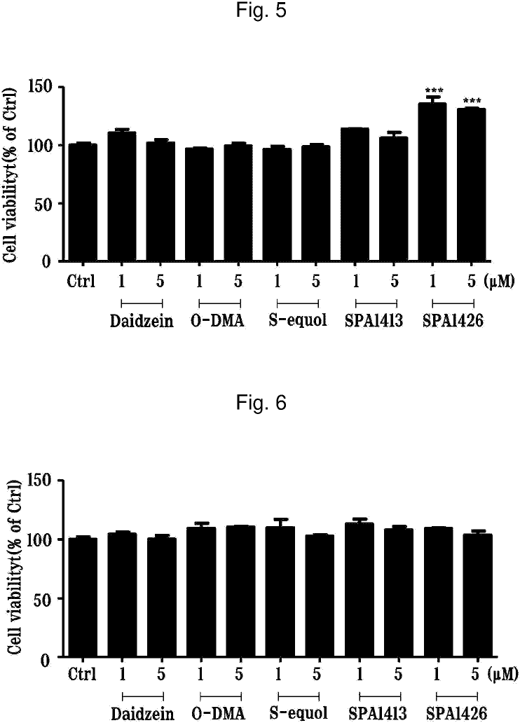

As can be seen from FIG. 6, each sample was found to exhibit no toxicity when applied at various concentrations.

### <2-4> Evaluation of Cytotoxicity in Human-Derived Neural Cells

Human-derived neural cells (N2a cells) purchased from the Korean Cell Line Bank were seeded at a density of 2x10⁵ cells/well into 96-well plates, each well containing DMEM (Dulbecco’s Modified Eagle’s Media) supplemented with 10% FBS and 1% antibiotics, and stabilized by incubation for 24 hours. Thereafter, the cells were treated for 24 hours with various concentrations (1 and 5 µM) of daidzein, o-desmethylangolensin, S-equal, SPA1413, and SPA1426.

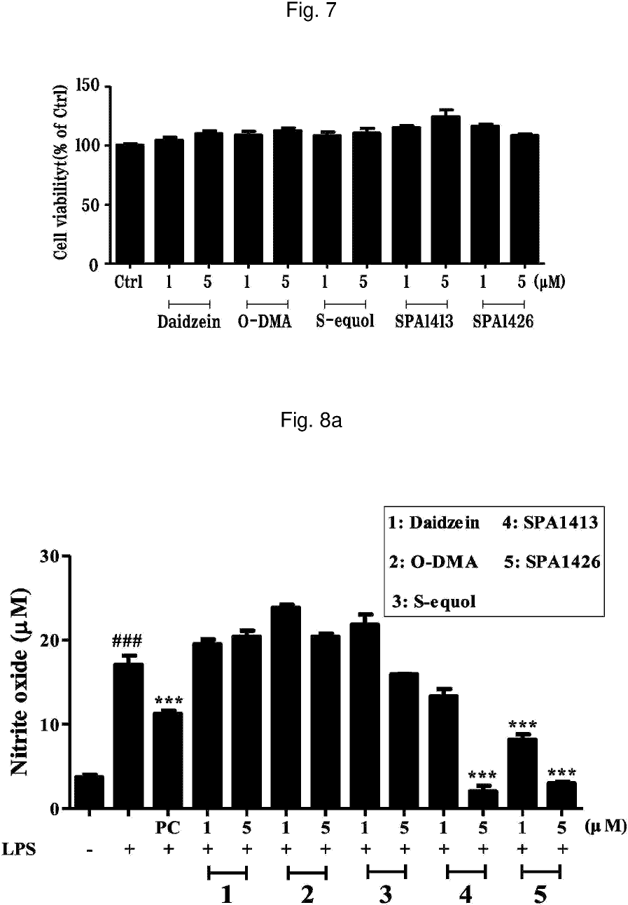

As can be seen from FIG. 7, each sample was found to exhibit no toxicity when applied at various concentrations.

The data obtained above imply that the compounds are cytoprotective without cytotoxicity.

### <Experimental Example 3> Anti-Neuroinflammatory Study Using Brain Cell Line

### <3-1> Assay for Nitric Oxide Production

BV2 cells were seeded at a density of 4 x 10⁵ cells/well into 96-well plates, and primary microglial cells isolated from the brain of 1-year-old ICR mice were seeded at a density of 4 x 10⁵ cells/well into 24-well plates.

After stabilization for 24 hours, the cells were treated with various concentrations (1 and 5 µM) of daidzein, o-desmethylangolensin, S-equal, SPA1413, and SPA1426 for 30 minutes and then with LPS (100 ng/ml) for 24 hours at 37° C. The culture media were collected and measured for NO production using a Griess reagent solution (1% sulfanilamide and 0.1% N-1-naphthyl ethylenediamine dihydrochloride in 5% phosphoric acid). Briefly, a total of 50 µL of the supernatant was transferred to new 96-well plates and mixed with the same volume of the Griess reagent solution, followed by measuring OD at 570 nm. In addition, the cell viability was assayed in the same manner as in Experimental Example 2.

As can be understood from data in FIG. 4 (lower), 8a, and 9a, treatment with LPS increased NO production while SPA 1413, SPA 1426, and SPA 1427 decreased NO production in dose-dependent manners.

In particular, the IC₅₀ value was significantly lower for the group treated with SPA 1413 and SPA 1426 than the group treated with LPS (Table 1)

TABLE 1

| | Compounds | IC₅₀ (µM) |
|---|---|---|
| 1 | Daidzein | 86.48 |
| 2 | O-DMA | 34.66 |
| 3 | S-equol | 11.70 |
| 4 | SPA1413 | 5.20 *** |
| 5 | SPA1426 | 3.67 *** |
| PC | L-NMMA (10 µM) | 15.12 |

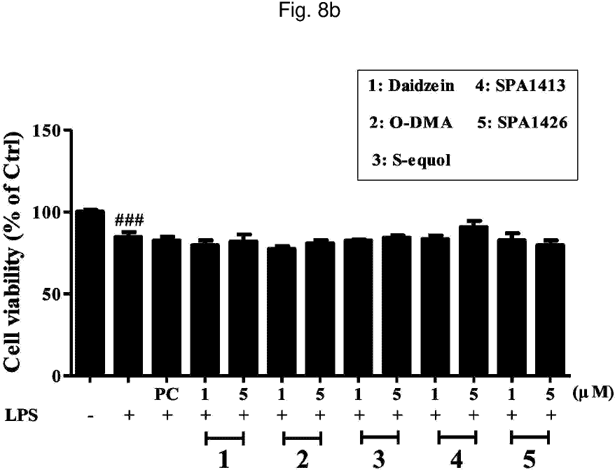

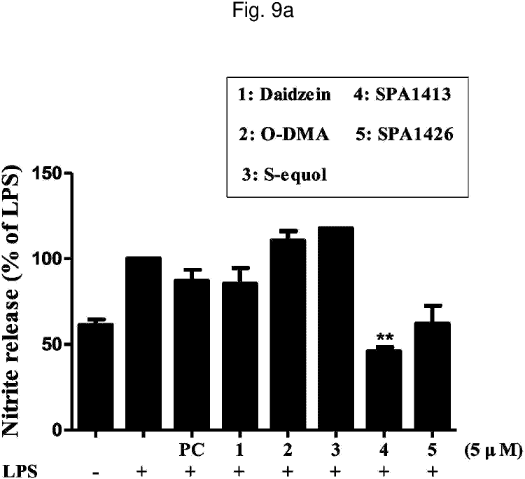

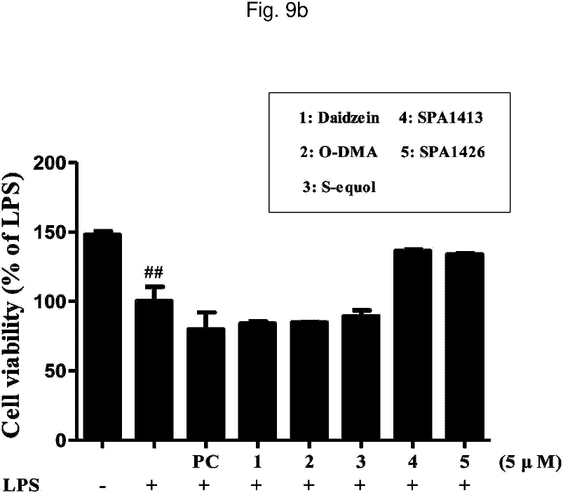

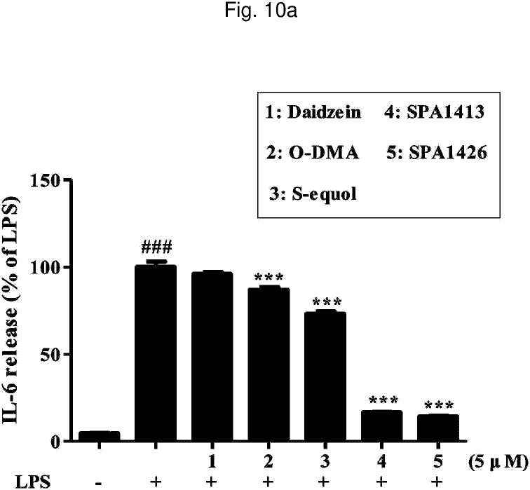

In addition, as shown in FIGS. 8b and 9b, the cell viability was increased in the group treated with SPA 1413 and SPA 1426 compared to the group treated with LPS.

### <3-2> Assay for IL-6 and TNF-a Production

BV2 cells were seeded at a density of 4x10⁵ cells/well into 96-well plates and stabilized for 24 hours. Thereafter, the cells were treated with 5 µM of daidzein, o-desmethylangolensin, S-equal, SPA1413, or SPA1426 for 30 minutes and then with LPS (100 ng/ml) for 24 hours at 37° C. The culture medium was collected and quantitatively measured for IL-6 and TNF-a released to the BV2 culture supernatant, using the enzyme immunoassay method Competitive Enzyme-Linked Immuno Assay (ELISA) kit (R&D systems, Minneapolis, MN, USA). The IL-6 and TNF-a releases from the groups treated with the samples were evaluated relative to that from the LPS-treated group, which was set to be 100%, and the results are depicted in FIGS. 10a and 10b.

As can be seen in FIGS. 10a and 10b, treatment with LPS increased IL-6 and TNF-6 production while SPA 1413 and SPA 1426 significantly decreased IL-6 and TNF-6 production.

### <3-3> Inhibitory Effect on COX-2 and iNOS Protein Expression

BV2 cells were aliquoted at a density of 1x10⁶ cells/well into 60 mm dishes and stabilized for 24 hours. Thereafter, the cells were treated with 5 µM of S-equal, SPA1413, or SPA1426 for 30 minutes and then with LPS (100 ng/ml) for 24 hours at 37° C. After being cultured to reach 100% confluence, the cells were washed with PBS and lysed with a lysis buffer (PRO-PREP™ Protein Extraction Solution, Intron Biotechnology, Seongnam, Korea). With bovine serum albumin (BSA) serving as a standard, the protein contents of the lysate supernatants were measured by Bio-Rad Protein Assay (Bio-Rad, California, USA) to determine the total protein content in each sample. Afterwards, each of the samples was loaded in a protein amount of 30 µg on a 10-12% SDS-PAGE gel and run for electrophoresis. The electrophorized proteins were transferred to a PVDF membrane which was then blocked with 5% skim milk and incubated with antibodies to COX-2 or iNOS (Cell Signaling Technologies, Massachusetts, USA). Detection was conducted with a ChemiDoc XRS+ imaging system (Bio-Rad, CA, USA).

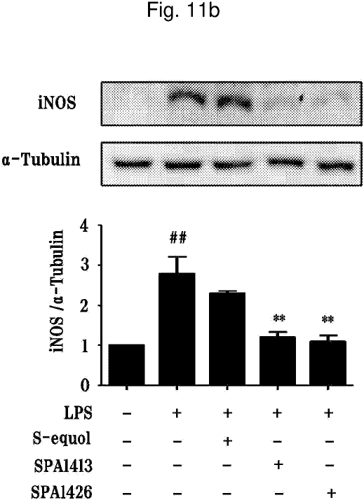

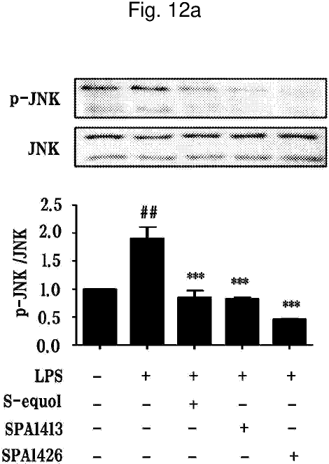

As can be seen in FIGS. 11a and 11b, the expression levels of COX-2 and iNOS were significantly increased by LPS, but significantly decreased by SPA 1413 and SPA 1426, compared to the control.

### <3-4> Inhibitory Effect on MAPKs (JNK and P38) Protein Expression

BV2 cells were aliquoted at a density of 1x10⁶ cells/well into 60 mm dishes and stabilized for 24 hours. Thereafter, the cells were treated with 5 µM of S-equal, SPA1413, or SPA1426 for 30 minutes and then with LPS (100 ng/ml) for 24 hours at 37° C. After being cultured to reach 100% confluence, the cells were washed with PBS and lysed with a lysis buffer (PRO-PREP™ Protein Extraction Solution, Intron Biotechnology, Seongnam, Korea). With bovine serum albumin (BSA) serving as a standard, the protein contents of the lysate supernatants were measured by Bio-Rad Protein Assay (Bio-Rad, California, USA) to determine the total protein content in each sample. Afterwards, each of the samples was loaded in a protein amount of 30 µg on a 10-12% SDS-PAGE gel and run for electrophoresis. The electrophorized proteins were transferred to a PVDF membrane which was then blocked with 5% skim milk and incubated with antibodies to JNK or p38 (Cell Signaling Technologies, Massachusetts, USA). Detection was conducted with a ChemiDoc XRS+ imaging system (Bio-Rad, CA, USA).

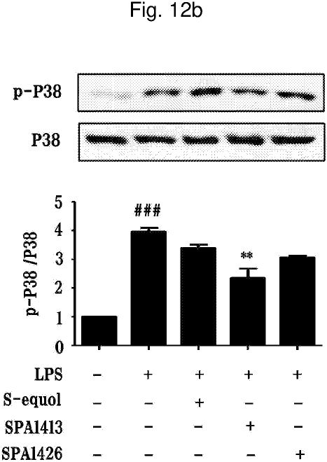

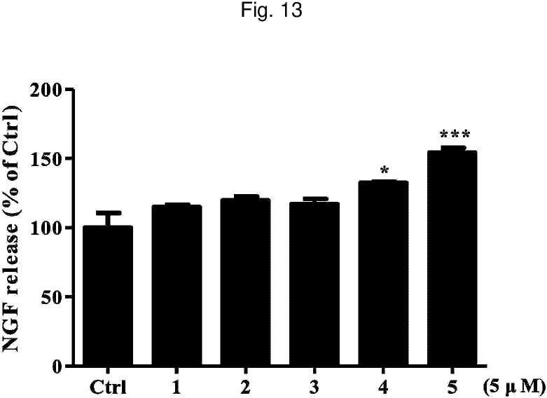

As can be seen in FIGS. 12a and 12b, the expression levels of JNK and P38 proteins were significantly increased by LPS, compared to the control, but were decreased in the groups treated with SPA 1413 and SPA 1426, compared to the LPS-treated group.

The data obtained above demonstrate an inhibitory effect of SPA1413, SPA1426, and SPA1427 on neuroinflammation factors, implying that the compounds of the present disclosure exhibit a neuroprotective activity by inhibiting the generation of inflammatory factors causative of neuroinflammation.

### <Experimental Example 4> Neuroprotective Effect Study

### <4-1> Assay for Nerve Growth Factor (NGF) Release in Rat-Derived Neuroglia

C6 cells were seeded at a density of 4x10⁵ cells/well into 96-well plates and stabilized for 24 hours. Thereafter, the cells were treated with 5 µM of daidzein, o-desmethylangolensin, S-equal, SPA1413, or SPA1426 for 24 hours. The culture medium was collected and quantitatively measured for NGF released to the C6 culture supernatant using the enzyme immunoassay method Competitive Enzyme-Linked Immuno Assay (ELISA) kit (R&D systems, Minneapolis, MN, USA). The NGF releases from the groups treated with the samples were evaluated relative to that from the normal group, which was set to be 100%, and the results are depicted in FIG. 13.

As can be seen from FIG. 13, the NGF content was significantly increased in the groups treated with SPA 1413 and SPA 1426 compared to the normal group.

### <4-2> Assay for Neurite Outgrowth in N2a Cells

N2a cells were aliquoted at a density of 1x10⁴ cells/well into 24-well plates, treated with daidzein, o-desmethylangolensin, S-equal, SPA1413 and SPA1426 at 5 µM, and evaluated for neurite outgrowth by the real-time cell monitoring system InCucyte. The cells were photographed every 2 hours, and the neurites were counted through Incucyte software.

As shown in FIGS. 14a and 14b, the neurite outgrowth was significantly increased by daidzein, SPA1413, and SPA1426. In particular, the morphological traits also exhibited that SPA1413 and SPA1426 increased neurite outgrowth, compared to the control.

### <4-3> Assay for Inhibitory Activity Against Beta Amyloid Oligomer in Primary Microglia

Primary microglial cells isolated from the brain of 1-year-old ICR mice were seeded at a density of 2x10⁵ cells/well into 96-well plates and stabilized for 24 hours. Thereafter, the cells were treated with 5 µM of daidzein, S-equal, SPA1413, or SPA1426 for 1 hour and then with beta amyloid oligomer (AB₁₋₄₀, 10 µM) for 24 hours at 37° C. Cell viability was assayed in the same manner as in Experimental Example 2.

As shown in FIG. 15, the group treated with Aβ₁₋₄₀ decreased in cell viability, compared to the control group. In particular, the group treated with SPA1413 significantly increased in cell viability.

The results indicate that the compounds of the present disclosure have an effect of protecting nerve cells by increasing the secretion of nerve growth factor, promoting the formation of axons, and inhibiting amyloid beta-induced apoptosis.

### <Experimental Example 5> Test for Inhibition Against Dementia Gene (PSEN1) Expression

### <5-1> Cytotoxicity (MTT Assay)

HEK293 cells and the HEK293 cells overexpressing the dementia gene PSEN1 were seeded at a density of 2x10⁵ cells/well into 96-well plates containing a DMEM medium and stabilized overnight, following by treating the cells with various concentrations (1, 5, and 10 µM) of the sample (SPA1413) for 24 hours. After removal of the culture medium, a 0.5 mg/ml MTT solution was added in an amount of 100 µl to each well and incubated at 37° C. for at least one hour in an incubator. The MTT was removed and 200 µl of DMSO was added to each well to dissolve the formazan. Absorbance was read at 540 nm on an ELISA reader to determine whether or the sample was toxic to the cells.

As can be seen from FIG. 16, the SPA1413-treated groups were the same as or almost similar to the normal control group (Control) in terms of cell viability. In particular, the PSEN1 gene-overexpressing HEK293 cell line was observed to increase in cell viability, compared to the normal cells, indicating that the compound of the present disclosure is not toxic to nerve cells and has a cytoprotective effect against the dementia gene-overexpressed cells.

### <5-2> Test for Inhibition Against Dementia Gene

HEK293 cells and PSEN1-overexpressed HEK293 cells were seeded at a density of 1x10⁶ cells/well into 6-well plates containing a DMEM medium and stabilized overnight, following by treating the cells with or without various concentrations (0.1 and 1 µM) of the sample (SPA1413) for 24 hours. Thereafter, RNA was extracted from the cells, using TRIzol reagent. Reverse transcription was performed using the GoScript™ reverse transcription system according to the manufacturer’s instructions. A qRT-PCR reaction was performed in a volume of 20 µl using the standard SYBR Green PCR kit and Roche Light® Instrument according to the standard protocol. Relative gene expression levels were calculated by the 2^{-ΔΔCt} method.

As can be seen in FIG. 17, treatment of the PSEN1 gene-overexpressed cells with SPA1413 inhibited the upregulated expression of PSEN1 gene, implying that the compound of the present disclosure can be a useful therapeutic agent for degenerative brain diseases, especially Alzheimer’s disease.

### <Experimental Example 6> Study for Efficacy for Breaking Down Methylglyoxal (MGO)-Induced Advanced Glycation End Products (AGEs)

Bovine serum albumin (BSA) was stored at 37° C. for 7 days in mixture with methylglyoxal (MGO) and sodium azide) to prepare AGEs. The AGEs derived from MGO are referred to as “MGO-AGEs”. MGO-AGEs with a concentration of 1 mg/ml were treated for 24 hours with various concentrations (0.1, 0.2, and 0.4 mM) of SPA1413. As a positive control, aminoguanidine (AG), known as an AGE inhibitor, was used. MGO-AGEs were reacted with 2,4,6-trinitrobenzene sulfonic acid (TNBSA) and 4% sodium bicarbonate, followed by adding 10% sodium dodecyl sulfate and 1 N HCl to terminate the reaction. Absorbance was read at 335 nm accounting for free amines, which resulted from the breaking degradation of AGEs, in a microplate to evaluate the efficacy of degrading AGEs.

It could be understood from data of FIG. 18 that SPA1413 of the present disclosure has an ability to degrade AGEs as increased levels of free amines were detected in the groups treated with SPA1413 of the present disclosure, compared to the negative control (MGO-AGEs).

In addition, referring to FIG. 19, higher levels of free amines were detected after treatment with SPA1426 and SPA1427 than SPA1413, implying that SPA1426 and SPA1427 have better ability to degrade AGEs than SPA1413.

### <Experimental Example 7> Cognitive Function Test

### <7-1> Preparation of Experimental Animals

The experiment was conducted with 5X-FAD transgenic mice having Alzheimer’s disease introduced therein. The 5X FAD mouse is an animal model of Alzheimer’s disease with all the human AD-linked mutations: Swedish (K670N/ M671N), Florida (1716V) and London (V7171) in APP (695) and M146L in Presenilin 1

5xFAD mice were purchased from Jackson Lab and bred in and in through mating with C57B16/SJL F1 females, based on the information provided by the supplier. Through genotyping, the mice were divided into groups of 7-9 members, including normal groups (WT) and genetically modified groups (5xFAD): 4.5-month-old 5xFAD mice (Tg) and age-matched controls (WT). To the WT or Tg groups, a vehicle or SPA1413 was orally administered at a dose of 10 mg/kg once a day for 1 month. To test whether SPA1413 has a therapeutic effect on Alzheimer’s disease, the mice in 5 groups (WT-vehicle; WT-SPA1413 5xFAD-vehicle; 5xFAD-SPA1413; and 5xFAD-donepezil) were evaluated for cognitive ability.

### <7-2> Novel Object Recognition Test

For a novel object recognition test, the Ethovision XT 9 system was used to examine whether an animal was interested in a new object. After an opaque box was made to establish an animal-rearing room-like environment therein, the experimental animals were acclimatized to the box for one day. An object newly recognizable to the animals was put into the box, and the time and frequency of staying near the object were measured to determine how interested the animals were in it. The effect of the drug was identified as a difference in cognitive abilities for new objects.

As can be seen in FIG. 20 (a to e), the object recognition test results and the memory index results were significantly different between the dSPA1413-administered 5xFAD mice (5xFAD-SPA1413) and the 5xFAD-saline group, but with no significant difference therebetween groups in terms of total distance and velocity. These results show that cognitive ability and spatial-related memory were increased by SPA1413 drug administration in 5xFAD mice.

### <7-3> Y-maze Test

Memory impairment was evaluated using the Y-shaped maze test. For the Y-shaped maze test, a Y-shaped arm was installed in a water tank. Cues were placed at the tips of the branches so that the animals could see the cues, and the animal’s movement was observed for 8 minutes through the Ethovision XT 9 system. After a random number was assigned to each arm, the arm number in which the animal entered was recorded. Memory was assessed by the animal’s ability to remember the route it had taken and to try to navigate the new route.

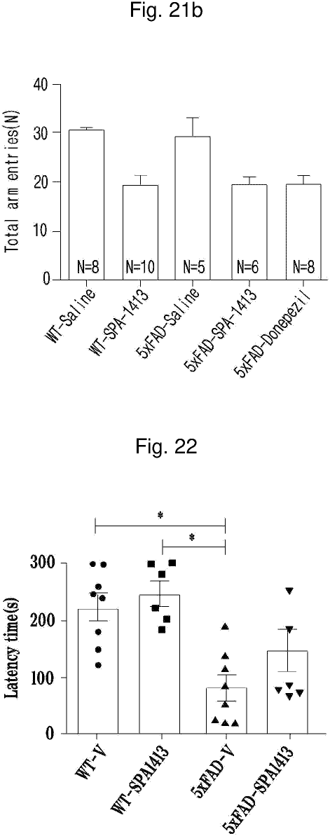

As understood from data of FIG. 21 (a and b), the SPA1413-administered 5xFAD mice (5xFAD-SPA1413) exhibited a significant difference in spontaneous alteration from the 5xFAD-saline group, but with no significant difference in total arm entries therebetween. These results show that spontaneous alteration ability, i.e., spatial memory, was increased by SPA1413 drug administration in 5xFAD mice.

### <7-4> Passive Avoidance Test

Passive avoidance test: Memory impairment was evaluated using the passive avoidance test. In the passive avoidance test, the experimental mice were taught an unpleasant stimulus (electric shock) from the outside, to evaluate the memory for the stimulus. In the training stage, the mice were taught to move to a dark room within 20 seconds after entering the Gemini according to the mice’s habit of preferring dark environments to bright environments (in order to learn unpleasant stimuli from the dark room the next day). After being trained to learn that an unpleasant stimulus was given thereto in the dark room, the mice were evaluated for memory for the corresponding content as step-through latency.

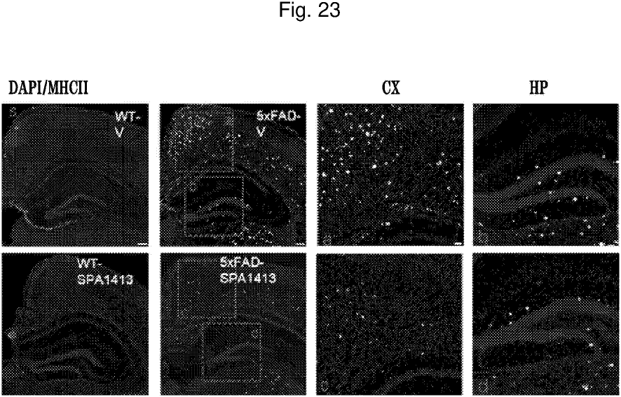

Referring to FIG. 22, the step-through latency of the 5xFAD-vehicle group was significantly reduced, compared with other groups, on the basis of the WT-saline group. In the SPA1413-administered 5xFAD mice, the step-through latency was significantly recovered. These results suggest that SPA1413 administration helps improve learning and memory ability in an animal model of Alzheimer’s disease.

### <7-5> Immunohistochemical Assay

After the animal behavior experiments, the brain tissues of the experimental animals were recovered, sectioned, and then stained with Thioflavin S for immunostaining chemistry using the 6E10 antibody.

Immunohistochemical staining is a technique for detecting specific proteins within tissues by staining the same. The brain tissues of the animals used in the behavioral experiments was recovered, and the hemisphere was fixed, frozen, and sectioned. Each slice was 22 µm thick. The sectioned brain tissue was stained with 6E10 antibody and Thioflavin S, and mounted on a slide glass using Vectashield. The stained tissues were observed through a fluorescence microscope, and the images are depicted in FIG. 23.

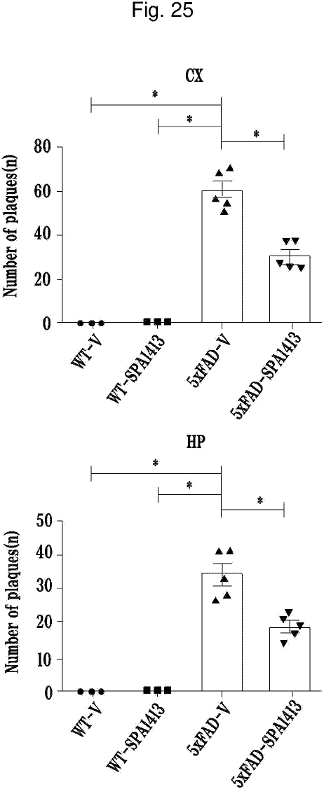

The quantitative analysis results of the images of the tissues stained with the 6E10 antibody and Thioflavin S are presented in FIGS. 24 and 25. Five experimental animals were selected from each group and analyzed. As a result, amyloid plaques were not found in the cortex and hippocampus of animals in the WT-saline group, which means that the animals used in the animal experiment corresponded to a histologically wild type. Amyloid plaques were observed in both cortex and hippocampus of the 5xFAD-vehicle and 5xFAD-SPA1413 group animals, which means that that the animal used in the animal experiment histologically corresponded to the Tg mice. However, amyloid plaques were significantly reduced in the SPA1413-administered 5xFAD mouse group, indicating that SPA1413 administration at a concentration of 10 mg/kg can reduce amyloid plaques in the cortex and hippocampus.

Activated microglia showing MHC II-positive reaction were not observed in the cortex and hippocampus of the WT-saline group animals, but found in the cortex and hippocampus of both the 5xFAD-vehicle and the 5xFAD-SPA1413 group animals. These results show that the neuroimmune response was increased in the Tg mice used for animal experiments. In contrast, the SPA1413-administered 5xFAD mouse group was observed to significantly decrease in the number of activated microglia showing a positive MHC II reaction. The data shows that administration of SPA1413 at a concentration of 10 mg/kg can significantly reduce neuroimmune responses in the cortex and hippocampus (FIGS. 26 and 27).

### <7-6> Western Blotting

After the animal behavior experiments, the brain tissues of the experimental animals were recovered, the brain tissues in the cortex and hippocampus regions of one hemisphere were separated, frozen, and stored until use. The tissues were thawed on the test day and treated with a lysis solvent, followed by protein isolation and quantitation. Thereafter, the same proteins were separated by SDS-PAGE according to molecular weight and treated with an anti-oligomeric Aβ antibody to identify a specific protein.

The images of the western blots analyzed for oligomeric Aβ proteins (60-70 kDa) in the cortex and hippocampus of 5xFAD-vehicle and 5xFAD-SPA1413 group animals are presented in FIG. 28, and the band intensity was analyzed and quantified using the image j program. The data indicate that the administration of SPA1413 at a concentration of 10 mg/kg to 5xFAD mice can significantly reduce the level of oligomeric Aβ proteins in the cortex and hippocampus.

Taken together, the data obtained above suggest that the compounds of the present disclosure can prevent, alleviate, or treat Alzheimer’s disease by protecting nerve cells and inhibiting aggregation of amyloid protein, and has the potential to improve cognitive function in patients with Alzheimer’s disease.

### <7-7> Weight Change

Mice in each group were monitored for weight change during the experimental period, and no significant differences were detected between the groups (FIG. 29).

### INDUSTRIAL APPLICABILITY

With the high activity of inhibiting fibrosis and oligomerization of Aβ and protecting nerve cells as well as improving cognitive functions, the compounds of the present disclosure, solvates thereof, hydrates thereof, or pharmaceutically acceptable salts thereof can be advantageously used for preventing, alleviating, or treating degenerative brain diseases, especially Alzheimer’s disease.

What is claimed is:

1. A composition, comprising a compound represented by the following Chemical Formula 1, a solvate thereof, a hydrate thereof, or a pharmaceutically acceptable salt thereof: 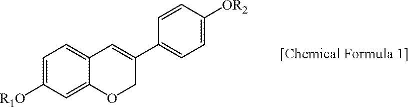
wherein,
R₁ and R₂ are each independently a hydrogen atom, or
a linear or branched alkyl carbonyl of C1-10.

2. The composition of claim 1, wherein R₁ and R₂ are each independently a hydrogen atom, or
a linear or branched alkyl carbonyl of C2-8.

3. The composition of claim 1, wherein R₁ and R₂ are each independently a hydrogen atom, 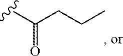 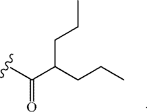
.

4. The composition of claim 1, wherein the compound represented by Chemical Formula 1 is any one selected from the group consisting of:
(1) 3-(4-hydroxyphenyl)-2H-chromen-7-ol;
(2) 4-(7-(butyryloxy)-2H-chromen-3-yl)phenyl butyrate);
(3) 4-(7-((2-ethylpentanoyl)oxy)-2H-chromen-3-yl)phenyl 2-ethylpentanoate.

5-13. (canceled)

14. A method for treating degenerative brain disease in a subject in need thereof, the method comprising:
administering to the subject a composition comprising a compound represented by the following Chemical Formula 1, a solvate thereof, a hydrate thereof, or a pharmaceutically acceptable salt thereof: 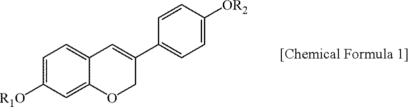
wherein,
R₁ and R₂ are each independently a hydrogen atom, or
a linear or branched alkyl carbonyl of C1-10.

15. The method of claim 14, wherein R₁ and R₂ are each independently a hydrogen atom, or a linear or branched alkyl carbonyl of C2-8.

16. The method of claim 14, wherein R₁ and R₂ are each independently a hydrogen atom, 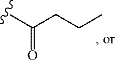 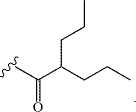
.

17. The method of claim 14, wherein the compound represented by Chemical Formula 1 is any one selected from the group consisting of:
(1) 3-(4-hydroxyphenyl)-2H-chromen-7-ol;
(2) 4-(7-(butyryloxy)-2H-chromen-3-yl)phenyl butyrate); and
(3) 4-(7-((2-ethylpentanoyl)oxy)-2H-chromen-3-yl)phenyl 2-ethylpentanoate.

18. The method of claim 14, wherein the degenerative brain disease is Alzheimer’s disease.
